(19) United States
(12) Patent Application Publication
(10) Pub. No.: US 2023/0078719 A1
(43) Pub. Date: Mar. 16, 2023

(54) CATHETER

(71) Applicant: TERUMO KABUSHIKI KAISHA, Shibuya-ku, Tokyo (JP)

(72) Inventors: Yuji MOTOSE, Kanagawa (JP); Daisuke SHIMADA, Shizuoka (JP)

(73) Assignee: TERUMO KABUSHIKI KAISHA, Shibuya-ku, Tokyo (JP)

(21) Appl. No.: 18/057,258

(22) Filed: Nov. 21, 2022

Related U.S. Application Data
(63) Continuation of application No. PCT/JP2021/012978, filed on Mar. 26, 2021.

(30) Foreign Application Priority Data
Jun. 1, 2020 (JP) 2020-095666

(51) Int. Cl. A61M 25/00

(52) U.S. Cl. CPC A61M 25/0014; A61M 2207/00

(57) ABSTRACT

A catheter includes a shaft and a hub that are firmly fixed to each other. The shaft is a tubular body in which a lumen extends throughout the shaft from a distal end to a proximal end. The shaft includes a shaft proximal surface at which the lumen opens and a shaft outer surface that is an outer peripheral surface of the tubular body. The hub is attached to the proximal end of the shaft. The hub includes a tubular accommodation unit configured to accommodate the shaft. The accommodation unit includes a hub melted surface directly welded to the shaft outer surface, and at least one of the accommodation unit and the hub is formed with a plurality of cavities at a position close to the hub melted surface.

### CROSS-REFERENCES TO RELATED APPLICATIONS

This application is a continuation of International Patent Application No. PCT/JP2021/012978 filed on Mar. 26, 2021, which claims priority to Japanese Patent Application No. 2020-095666 filed on Jun. 1, 2020, the entire content of both of which is incorporated herein by reference.

### TECHNOLOGICAL FIELD

The present invention generally relates to a catheter.

### BACKGROUND DISCUSSION

In recent years, treatment of the inside of a lumen such as a blood vessel by using a catheter has been actively performed since surgical invasiveness thereof is very low. The catheter generally includes a shaft including a lumen extending the shaft from a distal end to a proximal end, and a hub disposed at the proximal end of the shaft. The hub is formed with a passage communicating with the lumen in order to connect to a syringe or the like.

As a method for fixing the proximal end of the shaft to the hub, an insert molding method, a bonding method using an adhesive, and the like are known.

### SUMMARY

In an insert molding method described in Japanese Patent Application Publication No. 10-180802 (JP-A-H10-180802), a shaft is disposed in an injection mold, a part of the shaft is pressed by a fixing pin, and a resin for a hub is injection-molded at a high temperature and a high pressure. Therefore, deformation of the shaft caused by the fixing pin or displacement of the shaft in a longitudinal axis direction may occur. The deformation of the shaft and the displacement in the axial center direction may cause a decrease in fixing strength between the shaft and the hub.

In addition, in a bonding method using an adhesive described in Japanese Utility Model Publication No. 63-17486 (JP-UM-B-S63-17486), if a gap between an outer diameter of a shaft and a lumen of a shaft accommodation unit of a hub is excessively small, the adhesive cannot flow therein, and the gap remains between the hub and the shaft, which may cause a decrease in fixing strength between the shaft and the hub. On the other hand, when the gap between the outer diameter of the shaft and the lumen of the shaft accommodation unit of the hub is excessively large, it is difficult to completely fill the gap between the hub and the shaft with the adhesive, and thus the fixing strength between the shaft and the hub may decrease.

The catheter disclosed here is capable of firmly fixing a shaft and a hub.

A catheter according to one aspect of the disclosure includes: a shaft that is a tubular body in which a lumen extending the shaft from a distal end to a proximal end is formed, and that includes a shaft proximal surface in which the lumen is opened and a shaft outer surface that is an outer peripheral surface of the tube body; and a hub that is attached to the proximal end of the shaft. The hub includes a tubular accommodation unit that is configured to accommodate a portion of the shaft, the accommodation unit includes a hub melted surface that is configured to be directly welded to the shaft outer surface, and at least one of the accommodation unit and the hub includes a plurality of cavities at a position close to the hub melted surface.

In the catheter configured as described above, the accommodation unit and the hub are melted so as to enter each other by cavities. Therefore, the shaft and the hub can be firmly fixed.

The accommodation unit may be formed with a plurality of hub cavities, which are cavities, at a position close to the hub melted surface. Accordingly, the hub melted surface of the accommodation unit has a complicated shape with irregularities and is welded to the outer surface of the shaft. Therefore, the shaft and the hub can be firmly fixed.

The shaft may be formed with a plurality of shaft cavities, which are cavities, at a position close to a shaft melted surface of the shaft outer surface welded to the hub melted surface. Accordingly, the shaft outer surface has a complicated shape with irregularities and is welded to the hub melted surface. Therefore, the shaft and the hub can be firmly fixed.

Both the hub and the shaft may include a plurality of the cavities, and the hub melted surface and the shaft melted surface of the shaft outer surface welded to the hub melted surface may have irregularities and may be welded so as to enter each other. Accordingly, the shaft and the hub can be fixed more firmly.

According to another aspect a catheter comprises a shaft possessing a proximal portion that terminates in a proximal end of the shaft, with the shaft being a tubular body that includes a lumen extending through the shaft in a distal direction from the proximal end of the shaft. The shaft also possesses a shaft inner surface and a shaft outer surface, with the shaft inner surface surrounding the lumen in the shaft. The catheter also includes a hub that possesses a proximal end and a distal end, wherein the hub includes a lumen extending throughout the hub from the proximal end of the hub to the distal end of the hub. The hub includes an accommodation unit, and the lumen in the hub includes one portion that extends throughout the accommodation unit. The accommodation unit possesses an inner surface and also possesses an outer surface, with the proximal portion of the shaft being positioned in the one portion of the lumen in the accommodation unit and the inner surface of the accommodation unit fused to the outer surface of the proximal portion of the shaft, with material forming the accommodation unit and material forming the shaft intermixing with one another in a manner connecting the shaft and the hub to one another. The catheter also includes a plurality of cavities that each include an inner surface surrounding a closed space. The cavities are: i) in the accommodation unit of the hub, with at least some of the cavities being positioned radially closer to the inner surface of the accommodation unit than the outer surface of the accommodation unit; or ii) in the proximal portion of the shaft, with at least some of the cavities being positioned radially closer to the outer surface of the shaft than the inner surface of the shaft.

Another aspect of the disclosure involves a method of securing a shaft of a catheter to a hub of the catheter. The method comprises: positioning a proximal portion of the shaft in an accommodation unit of the hub so that there exists an axially overlapping portion in which the proximal portion of the shaft axially overlaps the accommodation unit of the hub, wherein the positioning of the proximal portion of the shaft in the accommodation unit of the hub comprises positioning the proximal portion of the shaft in the accommodation unit of the hub so that the inner surface of the accommodation unit of the hub faces the shaft outer surface of the proximal portion of the shaft and a portion of the shaft extends distally beyond a distal end of the accommodation unit. The method also includes heating the axially overlapping portion so that the outer surface of the proximal portion of the shaft and the inner surface of the accommodation portion are melted and fused together, with a plurality of cavities that each include an inner surface surrounding a closed space, wherein the cavities are: i) in the accommodation unit of the hub, with at least some of the cavities being positioned radially closer to the inner surface of the accommodation unit than the outer surface of the accommodation unit; or ii) in the proximal portion of the shaft, with at least some of the cavities being positioned radially closer to the inner surface of the shaft than the outer surface of the shaft..

### BRIEF DESCRIPTION OF THE DRAWINGS

FIG. 1 is a plan view illustrating a catheter according to an embodiment.

FIG. 2 is a cross-sectional view illustrating a hub and a proximal portion of a shaft.

FIG. 3 is a cross-sectional view illustrating a distal portion of the hub and the proximal portion of the shaft.

FIG. 4 is a cross-sectional view illustrating the distal portion of the hub before the shaft is fixed.

FIGS. 5A and 5B are cross-sectional views illustrating a process of welding the shaft to the hub, in which FIG. 5A illustrates a state in which welding is started, and FIG. 5B illustrates a state in which welding is in progress.

### DETAILED DESCRIPTION

Set forth below with reference to the accompanying drawings is a detailed description of an embodiment of a catheter representing an example of the new catheter disclosed here. The dimensions or scales on the drawings may be exaggerated or different from actuality/reality for convenience of description and illustration. In the following description, a side on which a catheter is operated will be referred to as a “proximal side”, and a side to be inserted into a living body will be referred to as a “distal side”.

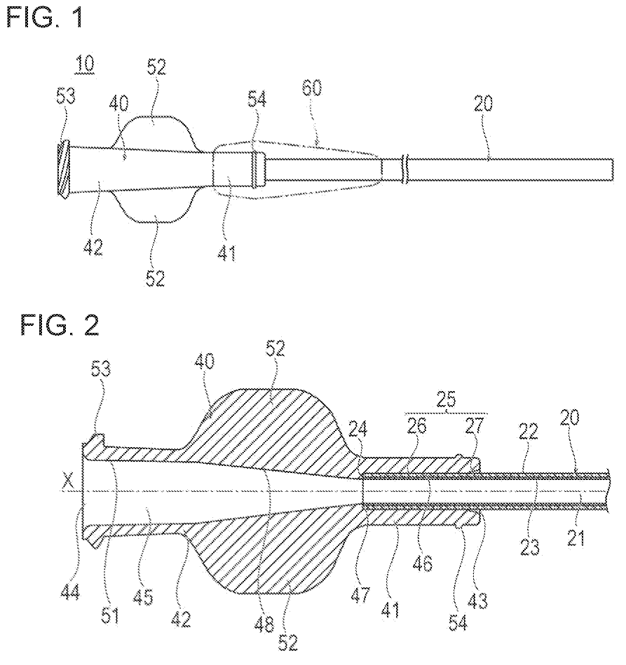

As illustrated in FIGS. 1 to 3, a catheter 10 according to one embodiment includes a shaft 20 that is an elongated tube body (tubular body), a hub 40 fixed to a proximal end of the shaft 20, and a flexible strain relief 60 configured to prevent bending of the shaft 20. In addition to a catheter that supports a guide wire, the catheter 10 may be a guiding catheter, an angiographic catheter, or a microcatheter, or may be a balloon catheter including an inflation lumen or an image diagnosis catheter. In addition, the catheter 10 may be an over-the-wire (OTW) catheter having a guide wire lumen extending from a distal end of the shaft to the hub, or may be a rapid exchange (RX) catheter in which the guide wire lumen is formed only in a distal portion of the shaft. For example, a guide wire lumen of an RX type balloon catheter is formed from the distal end of the shaft to an opening portion in the middle in the longitudinal axis direction of the shaft. An inflation lumen through which a fluid for inflating a balloon of the RX type balloon catheter flows is also provided and extends from the balloon to a hub at a proximal end of the catheter.

The shaft 20 is formed with a lumen 21 that extends throughout the shaft 20 from a distal end to the proximal end. The shaft 20 includes a shaft outer surface 22, a shaft inner surface 23, and a shaft proximal surface 24 (shaft proximal end surface).

The shaft outer surface 22 is an outer surface in a radial direction of the shaft 20, which is a tubular body, and extends from the distal end to the proximal end of the shaft 20. The shaft outer surface 22 includes a shaft proximal side outer surface 25 extending from the proximal end toward the distal end of the shaft 20 to a predetermined position along the length of the shaft 20. The shaft proximal side outer surface 25 is surrounded by and accommodated in the hub 40. The shaft proximal side outer surface 25 includes a substantially uniform outer diameter along the longitudinal axis X of the shaft 20. The shaft proximal side outer surface 25 includes a shaft melted surface 26 (fused) to the hub 40, and a shaft separated surface 27 that is disposed on the distal side of the shaft melted surface 26 and separated from the hub 40. The shaft separated surface 27 is not welded to the hub 40 and is separated from the hub 40 with a gap therebetween (i.e., a gap between the shaft separated surface 27 and the inner surface of the hub 40). A plurality of shaft cavities 32 are formed in the vicinity of the shaft melted surface 26 of the shaft 20 after the shaft 20 and hub 40 are welded or fused to one another. Each of the shaft cavities 32 includes an inner surface surrounding a closed space.

The shaft inner surface 23 is an inner surface in the radial direction of the shaft 20, which is a tubular body, and extends from the distal end to the proximal end of the shaft 20.

The shaft proximal surface or shaft proximal end surface 24 is a surface facing the proximal side (proximal direction) at the proximal end of the shaft 20, and is formed by being cut perpendicularly to the axial center X of the shaft 20.

The shaft 20 in the present embodiment includes an inner layer 28 that forms the shaft inner surface 23, an outer layer 29 that forms the shaft outer surface 22, and a reinforcement body 30 that is embedded in the shaft 20.

In addition to a polyamide resin, a polyester resin, a polyolefin resin and a polyurethane resin, examples of a constituent material from which the outer layer 29 may be fabricated include a polyamide elastomer, a polyester elastomer, a polyurethane elastomer, a mixture of one or more of these examples, and a mixture of materials having different hardnesses. The outer layer 29 may be formed by arranging materials having different hardnesses so as to become softer from the proximal end toward the distal end.

A constituent material from which the inner layer 28 may be fabricated may be the same material as the constituent material of the outer layer 29 described above, or may be a material different from the constituent material of the outer layer 29. The constituent material of the inner layer 28 may be a fluorine-based resin material such as a polytetrafluoroethylene resin in order to improve sliding property of an inner peripheral surface of the shaft 20.

The reinforcement body 30 reinforces the shaft 20, and is formed by braiding a plurality of reinforcement wires 31 into a tubular shape. In addition, the reinforcement body 30 may also be formed by spirally winding one or more reinforcement wires 31. The material of the outer layer 29 or the inner layer 28 enters gaps between the plurality of reinforcement wires 31 in the reinforcement body 30. The reinforcement wire 31 is made of a metal such as stainless steel or NiTi.

The hub 40 includes a tubular accommodation unit 41 that is disposed on the distal side and accommodates a proximal portion of the shaft 20, a hub main body 42 that is disposed on a proximal side of the accommodation unit 41, wings 52, a threading projection 53, and an annular projection 54. In the hub 40, a hub lumen 45 is formed which extends from a hub distal end opening 43 formed at a distal end of the accommodation unit 41 to a hub proximal end opening 44 formed at a proximal end of the hub main body 42. The hub lumen 45 includes an accommodation surface 46 that is an inner peripheral surface of the accommodation unit 41, an adjacent surface 47 that faces the shaft proximal surface 24, and a hub passage 48 that is an inner peripheral surface of the hub main body 42.

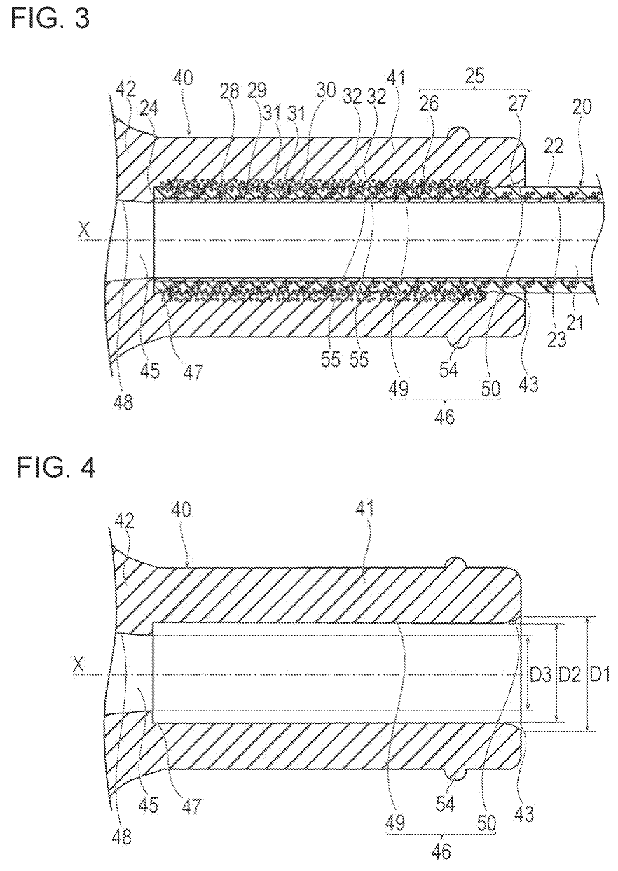

The accommodation surface 46 includes a hub melted surface 49 directly welded to the shaft melted surface 26 of the shaft proximal side outer surface 25, and a hub separated surface 50 that is separated outward in the radial direction from the shaft separated surface 27 and faces the shaft separated surface 27. That is, the hub separated surface 50 gradually expands in inner diameter as shown in FIGS. 3 and 4. The hub melted surface 49 extends from a proximal end of the accommodation surface 46 toward a distal direction. A proximal end of the hub melted surface 49 is connected to the adjacent surface 47. The hub separated surface 50 extends in the distal direction from a distal end of the hub melted surface 49. A gap between the hub separated surface 50 and the shaft proximal side outer surface 25 in the radial direction widens toward the distal direction. It should be noted that the hub separated surface 50 that forms the gap with the shaft proximal side outer surface 25 may not be provided. A plurality of hub cavities 55 are formed in the vicinity of the hub melted surface 49 of the accommodation unit 41 after the shaft 20 and hub 40 are welded or fused to one another. Each of the hub cavities 55 includes an inner surface surrounding a closed space. As shown in FIG. 3, the hub cavities may be provided so that all of the hub cavities 55 are positioned proximal of the gradually expanding hub separated surface 50 of the hub 40 (accommodation unit 41).

The adjacent surface 47 is an annular surface facing the distal side (distal direction), and is formed substantially perpendicular to the axial center X (center axis) of the shaft 20. An outer side of the adjacent surface 47 in the radial direction is connected to the hub melted surface 49. An inner side of the adjacent surface 47 in the radial direction is connected to a distal end of the hub passage 48.

The hub passage 48 extends from the adjacent surface 47 in a proximal direction. The hub passage 48 is formed in a tapered shape whose inner diameter gradually increases toward the proximal direction. The hub passage 48 is preferably coaxial with the accommodation surface 46 and further coaxial with the lumen 21. An inner diameter of the distal end of the hub passage 48 is preferably substantially equal to an inner diameter of the shaft 20, though is not limited in that regard. A part of the tapered hub passage 48 may include a Luer tapered portion 51 connectable to a syringe. A guide wire or a treatment catheter inserted from the hub proximal end opening 44 smoothly passes through the hub lumen 45 in the hub 40 and the lumen 21 in the shaft 20, and protrudes from a distal end of the catheter 10. Accordingly, the guide wire and the treatment catheter 10 can easily reach a target position such as a lesion area.

The wings 52 are formed so as to protrude from two opposing locations on an outer peripheral surface of the hub main body 42 such that an operator can easily grip and operate the hub 40. The threading projection 53 is formed on the outer peripheral surface of the hub main body 42 on the proximal side. The threading projection 53 can be engaged with a Luer lock type syringe or the like. The annular projection 54 is a projection formed over 360° on an outer peripheral surface of the accommodation unit 41. The annular projection 54 is fittable into a groove formed in an inner peripheral surface of the strain relief 60.

A constituent material from which the hub 40 may be fabricated is not particularly limited as long as the material is a thermoplastic resin that can be injection-molded, a material that easily transmits heat or electromagnetic waves is preferable, and specific examples thereof include a polyolefin resin, a polyamide resin, a polycarbonate resin, and a polyester resin.

Next, a method of welding the shaft 20 and the hub 40 will be described. As illustrated in FIG. 4, in the hub 40 before the shaft 20 is welded, an inner diameter of the accommodation surface 46 is larger on the distal side than on the proximal side. Specifically, an inner diameter D1 of the hub distal end opening 43 is larger than an inner diameter D2 of the distal end and the proximal end of the hub melted surface 49. In addition, an inner diameter D3 of the distal end of the hub passage 48 is smaller than the inner diameter D2 of the proximal end of the hub melted surface 49. The inner diameter D2 of the hub melted surface 49 is substantially equal to an outer diameter of the shaft proximal side outer surface 25. The inner diameter D1 of the hub distal end opening 43 is, for example, 0.92 mm. The inner diameter D2 of the proximal end of the hub melted surface 49 is, for example, 0.88 mm. The inner diameter D3 of the distal end of the hub passage 48 is, for example, 0.57 mm.

First, the proximal side of the shaft 20 is inserted into the accommodation unit 41, and the shaft proximal surface 24 is abutted against the adjacent surface 47. It should be noted that the shaft proximal surface 24 may not be abutted against the adjacent surface 47 and there may be a gap between the shaft proximal surface 24 and the adjacent surface 47. In addition, a proximal end of the shaft proximal side outer surface 25 is abutted against the proximal end of the hub melted surface 49. It should be noted that the shaft proximal side outer surface 25 may not be abutted against the hub melted surface 49, and there may be a gap between the shaft proximal side outer surface 25 and the hub melted surface 49. As shown in FIG. 3, the portion of the lumen in the tubular accommodation unit may have an axial extent (length) greater than the axial extent (length) of the portion of the shaft 20 at which the shaft cavities 32 are provided.

Next, a mandrel is inserted into the lumen 21 of the shaft 20, and the shaft proximal side outer surface 25 and the accommodation unit 41 of the hub 40 are heated. That is, an axially overlapping portion in which the proximal portion of the shaft 20 axially overlaps the accommodation unit 41 of the hub 40 is heated. Accordingly, the shaft proximal side outer surface 25 and the accommodation surface 46 are melted, and the hub melted surface 49 and the shaft melted surface 26 are welded (fused). The hub melted surface 49 and the shaft melted surface 26 may have an integrated structure by being mixed with each other. A heating method is not particularly limited, and examples thereof include a method of emitting electromagnetic waves having a wavelength that allows the electromagnetic waves to be transmitted through the hub 40 and does not allow the electromagnetic waves to be transmitted through the shaft outer surface 22. Since the shaft outer surface 22 does not transmit the electromagnetic waves, the shaft proximal side outer surface 25 is heated and melted at first. Then heat of the shaft proximal side outer surface 25 is transferred to the accommodation unit 41 to melt the accommodation unit 41.

The electromagnetic waves include infrared rays in addition to heat, microwaves, and visible light. The infrared rays are near-infrared rays having a wavelength of about 0.7 µm to 2.5 µm, mid-infrared rays having a wavelength of about 2.5 µm to 4 µm, or far-infrared rays having a wavelength of about 4 µm to 1000 µm, and the infrared rays may be near-infrared rays, mid-infrared rays, far-infrared rays alone or containing two or more types thereof, and may also contain visible light or microwaves.

An electromagnetic wave irradiation method is not particularly limited, and a semiconductor solid-state laser such as a YAG laser using neodymium, a fiber laser, or the like may be used.

The term “electromagnetic waves are transmitted” means that, in addition to being transparent to the naked eye under visible light, a measured transmittance (hereinafter, referred to as the transmittance) is 80% or more, and more preferably 85% or more. The transmittance can be measured by irradiating a sheet having a thickness of 0.4 mm to 0.5 mm prepared by melt-pressing resin pellets with electromagnetic waves having a specific wavelength and using a spectroscopic analyzer, for example, a Fourier transform infrared and near-infrared spectroscopic analyzer. Therefore, since the electromagnetic waves are not limited to visible light, the term “electromagnetic waves are transmitted” includes being transparent with respect to a specific wavelength even if the electromagnetic waves are colored or opaque to the naked eye.

In addition, the term “electromagnetic waves are not transmitted” means that, in addition to being opaque or colored to the naked eye under visible light, the transmittance is less than 80%, preferably less than 10%, and more preferably less than 1%. Therefore, since the electromagnetic waves are not limited to visible light, the term “electromagnetic waves are not transmitted” includes being opaque or absorbed with respect to a specific wavelength even if the electromagnetic waves are transparent to the naked eye.

In the outer layer 29, a pigment that does not transmit heat or electromagnetic waves, or a pigment that absorbs heat or electromagnetic waves, may be mixed in an amount of 0.01 wt% or more and less than 10 wt%, preferably 0.05 wt% or more and 5 wt% or less, and more preferably 0.1 wt% or more and 1 wt% or less with respect to total resin. Alternatively, the outer layer 29 may not contain any pigment, contrast agent, or the like, and the resin forming the outer layer 29 may have a low transmittance with respect to a specific wavelength. Alternatively, in the outer layer 29, a metal having X-ray contrast properties may be mixed in place of, or together with the pigment.

The pigment is not particularly limited as long as the pigment is a pigment that develops white, black, blue, red, or yellow or a mixture thereof, and a black pigment, for example, carbon black is preferable as a pigment that easily absorbs electromagnetic waves. The X-ray contrast agent is, for example, a compound of gold, bismuth, and tungsten, and is more preferably in the form of powder.

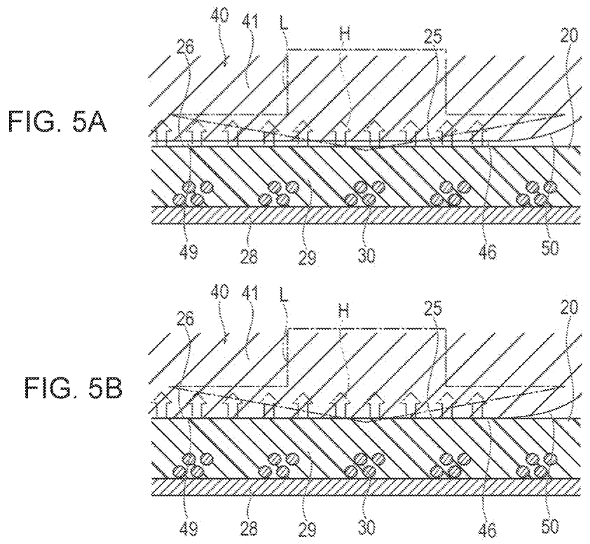

For example, as illustrated in FIG. 5A, when the shaft 20 and the hub 40 are welded to each other by irradiation with an infrared laser L, electromagnetic waves transmitted through the hub 40, which is transparent to a wavelength of the irradiated infrared laser L, are absorbed by the opaque resin, pigment, or the like of the outer layer 29 of the shaft 20 and mainly generate heat. Accordingly, the resin of the outer layer 29 melts and transfers heat H to the accommodation unit 41 of the hub 40, and at least a part of the accommodation surface 46 melts. As illustrated in FIG. 5B, a diameter of the melted accommodation surface 46 decreases, and the accommodation surface 46 is brought into close contact with the shaft proximal side outer surface 25, and is welded to the shaft proximal side outer surface 25. Accordingly, the hub melted surface 49 and the shaft melted surface 26 are welded and formed. At this time, the accommodation surface 46 melts and the diameter thereof decreases, and the outer peripheral surface of the accommodation unit 41 hardly melts and hardly deforms since heat is hardly transmitted. Therefore, due to the diameter decrease of the accommodation surface 46, a material constituting the accommodation surface 46 flows so as to fill a gap between the accommodation surface 46 and the shaft proximal side outer surface 25, and as a result, a length of the accommodation unit 41 along the axial center X is reduced. Accordingly, the accommodation unit 41 and the shaft proximal side outer surface 25 are favorably welded without any gap therebetween. In addition, the shaft proximal surface 24 and the adjacent surface 47 are also melted and welded by the heat generation of the outer layer 29. It should be noted that the shaft proximal surface 24 may not be welded to the adjacent surface 47.

When the shaft proximal side outer surface 25 and the accommodation surface 46 are welded or fused, as illustrated in FIG. 3, the shaft cavities 32 are formed in the vicinity of the shaft melted surface 26 of the shaft 20, and the hub cavities 55 are formed in the vicinity of the hub melted surface 49 of the accommodation unit 41. The cavities are generated by evaporation of materials, mixing of air, or the like. By forming the shaft cavities 32 and the hub cavities 55, the shaft melted surface 26 and the hub melted surface 49 have irregularities and are mixed with each other in a complicated manner, and are formed to enter each other and blend together. Therefore, the hub 40 and the shaft 20 are fixed to each other with a high coupling force. The hub melted surface 49 and the shaft melted surface 26 may have an integrated structure by being mixed with each other. It should be noted that only one of the shaft cavities 32 and the hub cavities 55 may be formed. That is, the shaft 20 and the hub 40 may be configured so that the shaft 20 includes shaft cavities 32 while the hub 40 does not include hub cavities, or the shaft 20 and the hub 40 may be configured so that the hub 40 includes hub cavities 55 while the shaft 20 does not include shaft cavities. In this case, the hub melted surface 49 and the shaft melted surface 26 are still mixed with each other in a complicated and thorough manner, and are still formed to enter each other. Therefore, the hub 40 and the shaft 20 are fixed to each other with a high coupling force.

The hub separated surface 50 located at the distal end of the hub melted surface 49 is not welded to the shaft separated surface 27, and a gap is maintained between the hub separated surface 50 and the shaft separated surface 27. When the entire hub melted surface 49 located on the proximal side of the hub separated surface 50 is welded to the shaft melted surface 26, the irradiation of the infrared laser L is stopped. Accordingly, fixation of the hub 40 and the shaft 20 is completed.

As described above, the catheter 10 according to the present embodiment is the catheter 10 including: the shaft 20 that is the tube body in which the lumen 21 extending the shaft 20 from the distal end to the proximal end is formed, and that includes the shaft proximal surface 24 in which the lumen 21 is opened and the shaft outer surface 22 that is the outer peripheral surface of the tube body; and the hub 40 attached to the proximal end of the shaft 20. The hub 40 includes the tubular accommodation unit 41 configured to accommodate the shaft 20, the accommodation unit 41 includes the hub melted surface 49 directly welded to the shaft outer surface 22, and at least one of the accommodation unit 41 and the hub 40 is formed with a plurality of cavities at a position close to the hub melted surface 49.

In the catheter 10 configured as described above, the shaft 20 and the hub 40 are welded so as to enter each other and become intermixed by the plurality of cavities (i.e., the material forming the shaft 20 and the hub 40 enter each other and become intermixed by the cavities). Therefore, the shaft 20 and the hub 40 can be firmly fixed together. Therefore, the shaft 20 can be prevented from being detached from the hub 40 in a case where a high pressure of a contrast agent injected into the catheter 10 is applied, a case where a tensile force acts between the hub 40 and the shaft 20 when the shaft 20 is pulled out from a body, or the like.

In addition, the accommodation unit 41 is formed with the plurality of hub cavities 55, which are cavities, at a position close to the hub melted surface 49. Accordingly, the hub melted surface 49 of the accommodation unit 41 has a complicated shape with irregularities and is welded to the shaft outer surface 22. Therefore, the shaft 20 and the hub 40 can be firmly fixed. As illustrated in FIG. 3, at least some of the hub cavities 55 are positioned closer (radially closer in the radial direction) to the hub melted surface or inner surface 49 of the hub than the outer surface of the hub 40.

In addition, the shaft 20 is formed with the plurality of shaft cavities 32, which are cavities, at a position close to the shaft melted surface 26 of the shaft outer surface 22 welded to the hub melted surface 49. Accordingly, the shaft outer surface 22 has a complicated shape with irregularities and is welded to the hub melted surface 49. Therefore, the shaft 20 and the hub 40 can be firmly fixed. As illustrated in FIG. 3, at least some of the shaft cavities 32 are positioned closer (radially closer in the radial direction) to the shaft melted surface or outer surface 26 of the shaft 20 than the inner surface of the shaft 20.

In addition, both the hub 40 and the shaft 20 include the plurality of cavities, and the hub melted surface 49 and the shaft melted surface 26 of the shaft outer surface 22 welded to the hub melted surface 49 have irregularities and are welded so as to enter each other (become intermixed with one another or blended together). Accordingly, the shaft 20 and the hub 40 can be fixed more firmly.

The present invention is not limited to the above-described embodiment, and various modifications can be made by those skilled in the art within the technical idea of the present invention. For example, the shaft 20 may be heated by high-frequency induction heating during which heating is performed by electromagnetic induction. An electromagnetically-induced conductor is, for example, the reinforcement body 30.

The detailed description above describes embodiments of a catheter and catheter manufacturing method representing examples of the catheter and catheter manufacturing method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

Reference Signs List

| | |
|---|---|
| 10 | catheter |
| 20 | shaft |
| 21 | lumen |
| 22 | shaft outer surface |
| 23 | shaft inner surface |
| 24 | shaft proximal surface |
| 25 | shaft proximal side outer surface |
| 26 | shaft melted surface |
| 27 | shaft separated surface |
| 28 | inner layer |
| 29 | outer layer |
| 30 | reinforcement body |
| 32 | shaft cavity (cavity) |
| 40 | hub |
| 41 | accommodation unit |
| 42 | hub main body |
| 43 | hub distal end opening |
| 44 | hub proximal end opening |
| 45 | hub lumen |
| 46 | accommodation surface |
| 47 | adjacent surface |
| 48 | hub passage |
| 49 | hub melted surface |
| 50 | hub separated surface |
| 55 | hub cavity (cavity) |
| 60 | strain relief |

What is claimed is:

1. A catheter comprising:
a shaft possessing a distal end and a proximal end, the shaft being a tubular body that includes a lumen extending from the distal end of the shaft to the proximal end of the shaft, the shaft including a shaft proximal end surface at the proximal end of the shaft, the lumen opening to the shaft proximal end surface at the proximal end of the shaft, the shaft also possessing a shaft outer surface that is an outer peripheral surface of the tubular body;
a hub attached to the proximal end of the shaft;
the hub including a tubular accommodation unit that accommodates a portion of the shaft,
the accommodation unit including a hub melted surface directly fused to the shaft outer surface; and
the accommodation unit or the shaft including a plurality of cavities at a position adjacent to the hub melted surface.

2. The catheter according to claim 1, wherein the plurality of cavities includes hub cavities in the hub, the accommodation unit possessing an outer surface, the hub cavities being located closer to the hub melted surface of the accommodation unit than the outer surface of the accommodation unit.

3. The catheter according to claim 2, wherein the shaft possesses a shaft inner surface, the plurality of cavities including a plurality of shaft cavities provided in the shaft, the shaft cavities being located closer to the shaft outer surface than the shaft inner surface.

4. The catheter according to claim 1, wherein the plurality of cavities includes cavities in the hub and cavities in the shaft, the hub melted surface and a shaft melted surface of the shaft outer surface that is welded to the hub melted surface having irregularities and being welded so as to enter each other.

5. The catheter according to claim 1, wherein the shaft possesses a shaft inner surface, the plurality of cavities including a plurality of shaft cavities in the shaft, the shaft cavities being located closer to the shaft outer surface than the shaft inner surface.

6. The catheter according to claim 1, wherein the hub includes a distal end, the plurality of cavities including hub cavities in the hub, all of the hub cavities being positioned proximal of the distal end of the hub.

7. The catheter according to claim 1, wherein the tubular accommodation unit includes a distal end portion that terminates at a distal end of the tubular accommodation unit, the hub melted surface in the distal end portion of the tubular accommodation unit gradually expanding toward the distal end of the tubular accommodation unit, the plurality of cavities including hub cavities provided in the hub, all of the hub cavities being positioned proximal of the distal end portion of the hub.

8. The catheter according to claim 1, wherein the hub includes a hub main body in addition to the tubular accommodation unit, the hub main body being connected to and positioned proximal of the tubular accommodation unit, the hub including a lumen that extends throughout both the hub main body and the tubular accommodation unit, the lumen in the hub including a first portion in the tubular accommodation unit and a second portion in the hub, the hub melted surface surrounding the first portion of the lumen, the second portion of the lumen possessing an inner diameter less than an inner diameter of the first portion of the lumen.

9. The catheter according to claim 8, wherein a proximal end of the first lumen in the tubular accommodation unit terminates in a radially extending adjacent surface, the plurality of cavities including a plurality of shaft cavities in the shaft along a longitudinally extending proximal portion of the shaft, the first portion of the lumen possessing a longitudinal extent that extends from the adjacent surface to a distal end of the tubular accommodation unit, the longitudinally extending proximal portion of the shaft at which the shaft cavities are located being shorter than the longitudinal extent of the first portion of the lumen.

10. A catheter comprising:
a shaft possessing a proximal portion that terminates in a proximal end of the shaft, the shaft being a tubular body that includes a lumen extending through the shaft in a distal direction from the proximal end of the shaft, the shaft also possessing a shaft inner surface and a shaft outer surface, the shaft inner surface surrounding the lumen in the shaft;
a hub that possesses a proximal end and a distal end, the hub including a lumen extending throughout the hub from the proximal end of the hub to the distal end of the hub;
the hub including an accommodation unit, the lumen in the hub including one portion that extends throughout the accommodation unit, the accommodation unit possessing an inner surface that surrounds the one portion of the lumen in the accommodation unit, the accommodation unit also possessing an outer surface, the proximal portion of the shaft being positioned in the one portion of the lumen in the accommodation unit with the inner surface of the accommodation unit fused to the outer surface of the proximal portion of the shaft in a manner connecting the shaft and the hub to one another;
a plurality of cavities that each include an inner surface surrounding a closed space, the plurality of cavities facilitating intermixing of the material of the shaft and the material of the hub; and
the plurality of cavities being: i) in the accommodation unit of the hub, with at least some of the cavities being positioned radially closer to the inner surface of the accommodation unit than the outer surface of the accommodation unit; or ii) in the proximal portion of the shaft, with at least some of the cavities being positioned radially closer to the inner surface of the shaft than the outer surface of the shaft.

11. The catheter according to claim 10, wherein the plurality of cavities are in both the accommodation unit and the shaft.

12. The catheter according to claim 10, wherein the plurality of cavities are in the accommodation unit of the hub, all of the cavities in the accommodation unit of the hub being proximal of the distal end of the hub.

13. The catheter according to claim 10, wherein the accommodation unit includes a distal end portion that terminates at the distal end of the accommodation unit, the inner surface of the accommodation unit in the distal end portion of the accommodation unit gradually expanding toward the distal end of the accommodation unit, the plurality of cavities being in the accommodation unit, all of the cavities in the accommodation unit of the hub being proximal of the distal end portion of the accommodation unit.

14. The catheter according to claim 10, further comprising a reinforcement body embedded in the shaft.

15. The catheter according to claim 14, wherein the plurality of cavities are in the shaft, the reinforcement body extending distally beyond the plurality of cavities.

16. The catheter according to claim 10, wherein the hub includes a hub main body in addition to the tubular accommodation unit, the hub main body being connected to and positioned proximal of the tubular accommodation unit, the lumen in the hub including a first portion of the lumen that is in the tubular accommodation unit and a second portion of the lumen that is in the hub, the inner surface of the accommodation unit of the hub surrounding the first portion of the lumen, the second portion of the lumen possessing an inner diameter less than an inner diameter of the first portion of the lumen.

17. The catheter according to claim 16, wherein a proximal end of the first lumen in the accommodation unit terminates in a radially extending adjacent surface, the plurality of cavities being in the proximal portion of the shaft, the first portion of the lumen possessing an axial extent that extends from the adjacent surface to the distal end of the accommodation unit, the proximal portion of the shaft at which the cavities are provided having an axial extent less than the axial extent of the first portion of the lumen.

18. A method of securing a shaft of a catheter to a hub of the catheter, the method comprising:
positioning a proximal portion of the shaft in an accommodation unit of the hub so that there exists an axially overlapping portion in which the proximal portion of the shaft axially overlaps the accommodation unit of the hub;
the positioning of the proximal portion of the shaft in the accommodation unit of the hub comprising positioning the proximal portion of the shaft in the accommodation unit of the hub so that the inner surface of the accommodation unit of the hub faces the shaft outer surface of the proximal portion of the shaft and a portion of the shaft extends distally beyond a distal end of the accommodation unit;
heating the axially overlapping portion so that there are a plurality of cavities that each include an inner surface surrounding a closed space and so that the outer surface of the proximal portion of the shaft and the inner surface of the accommodation portion are melted and fused together, the plurality of cavities being: i) in the accommodation unit of the hub, with at least some of the cavities being positioned radially closer to the inner surface of the accommodation unit than the outer surface of the accommodation unit; or ii) in the proximal portion of the shaft, with at least some of the cavities being positioned radially closer to the outer surface of the shaft than the inner surface of the shaft.

19. The method according to claim 18, wherein the plurality of cavities are in both the accommodation unit of the hub and the proximal portion of the shaft.

20. The method according to claim 19, wherein the cavities in the proximal portion of the shaft are closer to the outer surface of the shaft than the inner surface of the shaft, and the cavities in the accommodation unit are closer to the inner surface of the accommodation unit than the outer surface of the accommodation unit.
